(19) United States
(12) Patent Application Publication
(10) Pub. No.: US 2010/0047321 A1
(43) Pub. Date: Feb. 25, 2010

(54) SILVER ANTIMICROBIAL COMPOSITION AND USE

(76) Inventors: David W. Sandford, Rochester, NY (US); Sidney J. Bertucci, Rochester, NY (US); David A. Mowers, Rochester, NY (US)

Correspondence Address: J. Lanny Tucker;Patent Legal Staff, Eastman Kodak Company, 343 State Street, Rochester, NY, 14650-2201, US

(21) Appl. No.: 12/194,560

(22) Filed: Aug. 20, 2008

(51) Int. Cl. A61K 33/38; A61K 9/14; A61P 31/00; A61K 9/70

(52) U.S. Cl. 424/443; 424/489; 424/618

(57) ABSTRACT

Aqueous silver-containing composition is designed for use as an antimicrobial agent on fibers and fabrics. This composition includes silver halide particles, gelatin, and an additive that includes an N-heterocyclic acid having a pKa of from about 4 to about 9. The additive improves the redispersibility and shelf-life of the composition.

### FIELD OF THE INVENTION

The present invention relates to aqueous silver halide compositions comprising gelatin and an additive to extend shelf-life. The additives comprise N-heterocyclic acids of a specific acidity range that improve the redispersability and the colloidal stability of the aqueous silver halide and gelatin dispersions following extended storage. This invention also relates to a method of coating fibers, fabrics, or substrates with this composition to provide antimicrobial properties to the coated articles.

### BACKGROUND OF THE INVENTION

The antimicrobial properties of silver have been known for several thousand years. The general pharmacological properties of silver are summarized in “Heavy Metals”—by Stewart C. Harvey and “Antiseptics and Disinfectants: Fungicides; Ectoparasiticides”—by Stewart Harvey in The Pharmacological Basis of Therapeutics, Fifth Edition, by Louis S. Goodman and Alfred Gilman (editors), published by MacMillan Publishing Company, NY, 1975. It is now understood that the affinity of silver ion to biologically important moieties such as sulfhydryl, amino, imidazole, carboxyl and phosphate groups are primarily responsible for its antimicrobial activity, that is, it's capability to destroy or inhibit the growth of microorganisms such as bacteria, yeast, fungi, and algae, as well as viruses.

One very important use of silver based antimicrobials is as coatings for textiles. Various methods are known in the art to render antimicrobial properties to a target fiber. It is also known in the art to use binders in applied coating antimicrobial compositions, as described for example in JP1996-209531A (or Japanese Patent 2,998,584B2 by Suga et al.) where an antimicrobial fiber is obtained by sticking a very small amount of antimicrobial fine particles (such as silver halide particles) to the surface of the fiber using a polymeric binder. This publication describes the use of the composition for up to 10 laundry washings of wool and cotton fabrics, demonstrating durability of the antimicrobial effect.

In addition, U.S. Pat. No. 6,716,895 (Terry) describes the use of hydrophilic and hydrophobic polymers and a mixture of oligodynamic metal salts in antimicrobial compositions having a water content of less than 50 weight %. The use of silver halide in an antimicrobial coating is also described in U.S. Pat. No. 5,848,995 (Walden).

It is well known in the photographic art that gelatin is a useful hydrophilic polymer for the production of photographic silver halide emulsions. For example, gelatin is generally present during the precipitation of silver chloride from its precursor salts as a natural polypeptide peptizer in amounts of greater than 1.5 weight % to impart colloidal stability to the silver halide particles. However, due to colloidal instability, these silver halide dispersions are aggressively mixed by a mechanical stirrer throughout the precipitation process.

When the resulting “emulsion” is coated to form photographic films and papers, gelatin is typically present in amounts greater than 3 weight % or more likely greater than 10 weight %. To minimize settling of the dense particles (for example, AgCl has a density of 5.56), silver halide dispersions are stirred aggressively (or pumped rapidly under high-shear conditions sufficient to prevent settling) throughout the coating and drying processes. In such applications, it is desired or even required that the gelatin is present in a sufficient amount to solidify or “gel” the composition to minimize settling of the silver halide particles in the coatings. For storage, these photographic emulsions are generally cooled to about 35° C. until they solidify and then they are kept at about 5-10° C. until they are used. Sufficient gelatin is present in the dispersions to form a three-dimensional network adequate to prevent settling of silver halide particles.

The high gelatin levels are themselves a source of bio-activity and it is common to add biostats or biocides to the emulsions to prevent its spoilage prior to coating operations.

A major challenge in the practical use of silver halide as an antimicrobial agent is overcoming the poor colloidal stability of aqueous dispersions of fine particles (less than about 1 μm diameter) of silver halide. Poor colloidal stability results in aggregation and settling of the particles. Settling and aggregation (sometimes referred to an agglomeration or flocculation) may occur during shipping or long-term storage in potentially warm (tropical) conditions. If the aggregation or agglomeration of particles is substantially irreversible, then only partial redispersal of the particles by the end-user will be possible. Inefficient or incomplete transfer of the material from its original container to the end-user's manufacturing equipment (for example, textile coating bath) may result. In addition, antimicrobial efficacy may be compromised as release of silver ions from agglomerated particles may be inhibited, uniformity of the distribution of silver containing particles across a substrate (for example, a textile fiber or fabric) may be compromised, and as such the cost to the end user may be increased if a greater amount of silver halide is then required to achieve the desired antimicrobial effect.

In addition to the challenge of redispersal during transfer between containers, the potential settling of silver halide particles in the end-users final manufacturing containers can lead directly to non-uniformity and waste.

To address the undesirable consequences of colloidal instability, Schroeder et al in U.S. Patent Application Publication 2006/0068024 (Schroeder et al.) describes the use of low amounts (less than 3%) of gelatin to provide antimicrobial silver halide compositions that do not substantially gel or solidify at 25° C. These free flowing compositions can be easily transferred and mixed with aqueous diluents or other addenda prior to use as an antimicrobial coating for yarn or fabric.

In another approach, WO2006/105669 (Tessier et al.) describes the use of alkylammonium halogenides as cationic surfactants to improve the colloidal stability and redispersability of metal-containing antimicrobial agents.

### Problem to be Solved

While the noted art provides some suggestions for overcoming the problem of colloidal instability in the silver halide compositions, there remains a need to provide aqueous silver halide dispersions comprising gelatin with extended shelf-life. In particular, improvements in the redispersability and the colloidal stability of aqueous silver halide dispersions comprising gelatin following extended storage are desired.

### SUMMARY OF THE INVENTION

This invention provides a composition comprising at least 50 weight % water, silver halide particles, gelatin, and an additive that includes one or more N-heterocyclic acids at least one of which has a pKa of from about 4 to about 9, wherein the composition is substantially free of organic solvents.

This invention also provides a method of providing an antimicrobial coating comprising:

coating a fiber, fabric, or substrate (such as a film) with the composition of this invention, and

drying the coated fiber, fabric, or substrate.

Thus, this invention can provide a fiber, fabric, or substrate (such as a film) having a dried antimicrobial coating provided from the composition of this invention.

The present invention provides an improvement in the redispersibility and colloidal stability of silver halide compositions (dispersions) containing gelatin. These compositions can be used as antimicrobial agents containing very low amounts of gelatin that are able to flow at ambient temperatures (typically 25° C.) and can be kept at ambient temperatures for storage and transport in a non-stirred or non-agitated state for up to several weeks. This provides a significant advantage to both the manufacturer and user of the composition to provide durable antimicrobial coatings for various articles such as yarns, fibers, fabric, or other textiles.

The advantages of this invention are provided by the presence of one or more specific N-heterocyclic acids as defined herein.

### DETAILED DESCRIPTION OF THE INVENTION

The composition of this invention generally comprises water in an amount of at least 50 weight % (typically at least 80 weight % or more likely at least 90 weight %), silver halide particles, gelatin, and the additive described below to extend shelf-life.

Any type of useful gelatin can be used in an amount wherein the composition does not substantially gel or solidify at 25° C. In practical terms the composition, when sold as a concentrate, must be able to flow at 25° C. and be easily mixed with an aqueous diluent or other addenda prior to use as an antimicrobial coating. The composition also encompasses a more diluted form that is suitable for dip, pad, or other types of coating. The composition is substantially free of organic solvents such that use of the composition in textile manufacturing operations is considered to be safe from an explosion-proof perspective by regulatory agencies. Thus, no organic solvent is intentionally added to the composition. The amount of gelatin is generally present in an amount of at least 0.001 weight %, and less than 1.3 weight % or typically at least 0.15 weight % and less than 1 weight % or even less than 0.75 weight %.

Gelatin is an amphoteric polyelectrolyte that has excellent affinity to a number of substrates. The gelatin used in the practice of this invention may be processed by any of the well-known techniques in the art including; alkali-treatment, acid-treatment, acetylated gelatin, phthalated gelatin or enzyme digestion. The gelatin may have a wide range of molecular weights and may include low molecular weight gelatins if it is desirable to raise the concentration of the gelatin in the inventive composition without solidifying the composition. The gelatin in the present invention is added in an amount sufficient to peptize the surface of the silver halide and some excess of gelatin will always be present in the water phase. The gelatin may eventually be cross-linked in order to improve the durability of the antimicrobial coating composition. For this purpose, the composition may include a gelatin crosslinker such as alum, formaldehyde and free aldehydes such as glutaraldehyde, bis(iminomethyl) ether salts, s-triazines, or diazines in conventional amounts. The gelatin crosslinker is generally kept separate from the rest of the composition until a short time prior to using the composition as a coating.

The silver halide particles may be of any shape and halide composition. The type of halide may include chloride, bromide, iodide as well as mixtures of two or more the halides. The silver halide particles may be, for example, silver bromide, silver iodobromide, bromoiodide, silver iodide, or silver chloride. In some embodiments, the silver halide particles are predominantly silver chloride such as pure silver chloride, silver bromochloride, silver iodochloride, silver bromoiodochloride, and silver iodobromochloride particles. By predominantly silver chloride, it is meant that the particles are greater than about 50 mole percent silver chloride. More likely, they are greater than about 90 mole percent silver chloride or greater than about 95 mole percent silver chloride. The silver halide particles may either be homogeneous in composition or the core region may have a different composition than the shell region of the particles. The shape of the silver halide particles may be cubic, octahedral, tabular or irregular. More silver halide properties may be found in “The Theory of the Photographic Process”, T. H. James, ed., 4th Edition, Macmillan (1977). In some embodiments, the silver halide particles have a mean equivalent circular diameter of less than 1 μm, or typically less 0.5 μm.

The silver halide particles and associated coating composition of the present invention are applied to a fiber or fabric in an amount sufficient to provide antimicrobial properties to the treated fiber for a minimum of at least 10 washes, more preferably 20 washes and most preferably after 30 washes in accordance with ISO 6330:2003. The amount of silver halide particles applied to the target fiber or textile fabric is determined by the desired durability or length of time of antimicrobial properties. The amount of silver halide particles present in the composition will depend on whether the composition is one being sold in a concentrated form suitable for dilution prior to coating or whether the composition has already been diluted for coating. General levels of silver salt particles (by weight percent) in the formulation are from about 10⁻⁷% to about 10%, or from about 0.0001% to about 1% or more likely from about 0.001% to 0.5%. In a concentrated format the composition generally comprises silver halide particles in an amount of from about 0.1 to about 20%, or from about 0.5 to about 10%, and more likely from about 1 to about 5% in diluted compositions and up to 8% in concentrated compositions. It is a desirable feature of the invention to provide efficient antimicrobial properties to the target substrate (such as a yarn, fiber, or textile fabric) at a minimum silver halide level to minimize the cost associated with the antimicrobial treatment while providing excellent uniformity and consistency.

The silver halide particles can be formed by reacting silver nitrate with halide in aqueous solution. In the process of silver halide precipitation one can add the gelatin to peptize the surface of the silver halide particles thereby imparting colloidal stability to the particles, see for example, Research Disclosure September 1997, Number 401 published by Kenneth Mason Publications, Ltd., Dudley Annex, 12a North Street, Emsworth, Hampshire PO10 7DQ, ENGLAND.

The storage stability additives used in the compositions of this invention are N-heterocyclic acids each having a pKa of from about 4 to about 9 or from about 4.5 to about 8.5, or from about 4.8 to about 8.0. Such acids are organic compounds having one or more acidic nitrogen-hydrogen bonds. For example, the N-heterocyclic acid can be of the azole class of compounds such as azoles, diazoles, triazoles, or tetrazoles. Examples of useful compounds include but are not limited to, tetraazaindene, bromo-tetraazaindene, S-methyl-tetraazaindene, urazole, uric acid, benzotriazole, methylbenzotriazole, and chloronitrobenzotriazole. Uric acid and methylbenzotriazole are particularly useful. pKa refers to the negative log base 10 of acid dissociation constant.

The N-heterocyclic acid additives are generally present in an amount of from about 0.1 to about 5 weight %, or from about 0.5 to about 2 weight % relative to the amount (weight) of silver in the composition. A determination of the optimum amount of additive so as to not interfere with other components of the composition while providing the desired shelf-life stability can be readily done with routine experimentation.

In addition to gelatin, minor amounts of a hydrophobic binder resin may be included in the composition to improve the adhesion and durability of the silver salt particles once applied to a fabric surface. Such hydrophobic binders are well known in the art and are typically provided as aqueous suspensions of polymer microparticles. Materials suitable for use as hydrophobic binders include acrylic, styrene-butadiene, polyurethane, polyester, polyvinyl acetate, polyvinyl acetal, vinyl chloride and vinylidine chloride polymers, including copolymers thereof. If present, the concentration of such hydrophobic binders is less than 5 weight %.

There may also be present optional components, for example, thickeners or wetting agents in the composition to aid in the application of the composition to a substrate. Examples of wetting materials include surface active agents commonly used in the art such as ethyleneoxide-propyleneoxide block copolymers, polyoxyethylene alkyl phenols, polyoxyethylene alkyl ethers, and the like. Compounds useful as thickeners include, for example, particulates such as silica gels and smectite clays, polysaccharides such as xanthan gum, polymeric materials such as acrylic-acrylic acid copolymers, hydrophobically modified ethoxylated urethanes, hydrophobically modified nonionic polyols, and hydroxypropyl methylcellulose.

Also of use in the compositions of the invention are agents to prevent latent image formation. Some silver salts are light sensitive and discolor upon irradiation of light. However, the degree of light sensitivity may be minimized by several techniques known to those who are skilled in the art. For example, storage of the silver halide particles in a low pH environment will minimize discoloration. In general, pH below 7.0 is desired and more specifically, pH below 4.5 is preferred. Another technique to inhibit discoloration involves adding compounds of elements, such as, iron, iridium, ruthenium, palladium, osmium, gallium, cobalt, and rhodium, to the silver halide particles. These compounds are known in the photographic art to change the propensity of latent image formation; and thus the discoloration of the silver salt. Additional emulsion dopants are described in Research Disclosure, February 1995, Volume 370, Item 37038, Section XV.B., published by Kenneth Mason Publications, Ltd., Dudley Annex, 12a North Street, Elmsworth, Hampshire PO10 7DQ, England.

While these various additives may be helpful, in some embodiments, the composition of this invention consists essentially of water, the silver halide particles, gelatin, and one or more of the noted N-heterocyclic acids, each having a pKa of from about 4 to about 9.

In some embodiments, the composition of this invention comprises at least 90 weight % water, silver chloride particles in an amount of from about 0.001 to about 5 weight %, gelatin in an amount of from about 0.15 to about 1 weight %, and an additive that includes one or both of uric acid and methylbenzotriazole in an amount of from about 0.5 to about 5 weight % based on the weight of silver in the composition, and this composition is substantially free of organic solvents.

The composition of this invention can be applied to any particular substrate (such as polymeric films, papers, or metal foils) and is not limited in its use, but application to fibers, textile fabric, or yarn including, exhaustively any natural or manufactured fibers, is particularly useful. Examples of natural fibers include, cotton (cellulosic), wool, or other natural hair fibers, for example, mohair and angora. Examples of manufactured fibers include synthetics, such as, polyester, polypropylene, nylon, acrylic, polyamide, or, regenerated materials such as cellulosics. The target fiber or yarn may include any number of chemistries or applications prior to, during or after the application of the antimicrobial composition of the invention including, for example, antistatic control agents, flame retardants, soil resistant agents, wrinkle resistant agents, shrink resistant agents, dyes and colorants, brightening agents, UV stabilizers, lubricants, or antimigrants.

The composition of this invention can be applied to the desired substrate using in any of the well know methods in art including but not limited to, pad coating, knife coating, screen coating, spraying, foaming, and kiss-coating. The components of the inventive composition can be delivered in a single dispersion but in some embodiments they may be delivered as a separately packaged two-part system having the silver halide particles, gelatin, water, and N-heterocyclic acid(s) as Part A, and Part B comprising an aqueous suspension of any optional hydrophobic binder, additional hydrophilic binders, or gelatin cross-linker. Part A demonstrates improved shelf-life with regard to redispersability and colloidal stability following long-term storage at ambient temperatures without stirring or agitation. The two parts may be combined prior to a padding or coating operation and exhibit colloidal stability for the useful shelf-life of the combined composition, typically on the order of hours to days.

The following examples are intended to demonstrate, but not to limit, the invention.

### EXAMPLES

### Preparation of Silver Chloride Dispersion:

Silver chloride grains were prepared by the following process:

To a reactor charged with 184 g of gelatin, 15 g of sodium chloride, and 6,490 g of water, 2.8 molar silver nitrate solution and 3 molar sodium chloride solution were added at 186 cc/min and 182 cc/min, respectively, over 16.2 minutes with vigorous stirring. The temperature of the reactor was maintained at 46.1° C. throughout the precipitation process. The solution was then washed under constant volume conditions with an ultra-filtration column to remove soluble salts, and then diluted with an equal weight of distilled water. Vigorous agitation was maintained throughout the process by stirring or rapid pumping of the dispersion. The final dispersion of silver chloride grains in gelatin and water consisted of 21.8 g gelatin per mole of AgCl, a total weight of 3.5 kg dispersion per mole of AgCl, constituting a gelatin weight percent of 0.62. The resulting silver chloride grains had a mean equivalent circular diameter of 0.2 μm.

### Silver Assay Method:

Samples were analyzed for silver content using an Automated Thioacetamide Titration system (ATT) to assay silver ion (Ag⁺) present in silver containing compounds by a potentiometric titration. Silver chloride grains, which have been solubilized in a solution of thiosulfate, sodium hydroxide and gelatin, are titrated with thioacetamide to precipitate silver sulfide. The end-point of the titration is monitored potentiometrically using a silver sulfide-coated silver billet indicator electrode.

Invention Examples 1-7 below demonstrate the efficacy of various N-heterocyclic acid additives in improving colloidal stability as reflected in reduced settling of aqueous silver chloride dispersions that are redispersed and tested for settling after unstirred storage in an oven at 40° C. for 5 days.

### Comparative Example 1

### Settling of Product Stored at 5° C. Since Precipitation

A 100 ml aliquot of the silver chloride dispersion prepared as described above was stored unstirred at 5° C. for several hours, then redispersed by stirring for 5 minutes at 40° C. To test for settling, the stirring was stopped and a 10 ml portion was removed from the top of the aliquot (time=0 sample).

Additional samples were taken in a similar manner from the unstirred aliquot at 10 minute, 30 minute, and 60 minute intervals. By comparing the theoretical silver chloride concentration (measured on a sample taken from the vigorously stirred dispersion at the conclusion of the precipitation), assuming a perfectly dispersed solution, with that determined by the ATT assay for each sample taken from the top of the aliquot, it is possible to determine the amount of AgCl product that had settled in a given time interval. The settling data for these samples are shown in TABLE I below.

### Comparative Example 2

### Settling of Product Stored at 40° C. for 5 Days

A portion of the silver chloride dispersion prepared as described above was stored unstirred in an oven at 40° C. for 5 days and then redispersed and tested for settling as described in Comparative Example 1. The settling data for these samples are shown in TABLE I below.

### Comparative Example 3

### Settling of pH Adjusted Product

A portion of the silver chloride dispersion prepared as described above was adjusted from its original pH of 4 to a pH of 5.6 with sodium hydroxide solution, and then redispersed and tested for settling as described in Comparative Example 1. The settling data for these samples are shown in TABLE I below.

### Comparative Example 4

### Settling of pH Adjusted Product Stored at 40° C. for 5 Days

A portion of the silver chloride dispersion prepared as described above was adjusted from its original pH of 4 to a pH of 5.6 with sodium hydroxide solution, stored unstirred in an oven at 40° C. for 5 days, and then redispersed and tested for settling as described in Comparative Example 1. The settling data for these samples are shown in TABLE I below. However, an objectionable degree of discoloration was observed after this pH adjusted sample was stored at 40° C. for 5 days.

### Comparative Example 5

### Settling of Diluted Product

A 65.8 ml portion of the silver chloride dispersion prepared as described above was dispersed by stirring at 40° C. for 5 minutes, diluted by the addition of 183.5 ml of distilled water, and stirred thereafter for 5 minutes at 40° C. An aliquot was cooled to 22° C. and tested for settling as described in Comparative Example 1. The settling data for these samples are shown in TABLE I below. The gelatin content of these samples was 0.16 weight %.

### Comparative Example 6

### Settling of Diluted Product Stored at 40° C. for 5 Days

Product was diluted as described in Comparative Example 5 and then stored unstirred in an oven at 40° C. for 5 days before redispersal and testing for settling as described in Comparative Example 1. The settling data for these samples are shown in TABLE I below. The gelatin content of these samples was 0.16 weight %.

### Comparative Example 7

### Settling of Product With Saccharin Stored at 40° C. for 5 Days

A 100.0 ml portion of the silver chloride dispersion prepared as described above was dispersed by stirring at 40° C. for 5 minutes, a 6.3 ml solution containing 0.055 g of saccharin was added, and the mixture stirred thereafter for 5 minutes at 40° C. The mixture was subsequently stored unstirred in an oven at 40° C. for 5 days before redispersal and testing for settling as described in Comparative Example 1. The settling data for these samples are shown in TABLE I below. The gelatin content of these samples was 0.58 weight %.

### Comparative Example 8

### Settling of Product With APMT Stored at 40° C. for 5 Days

A 65.8 ml portion of the silver chloride dispersion described above was dispersed by stirring at 40° C. for 5 minutes, a 183.5 ml solution containing 0.036 g of acetamide, N-(3-(2,5-dihydro-5-thioxo-1H-tetrazol-1-yl)phenyl)-(APMT) was added, and the mixture was stirred thereafter for 5 minutes at 40° C. The mixture was subsequently stored unstirred in an oven at 40° C. for 5 days before redispersal and testing for settling as described in Comparative Example 1 was attempted. However, it was not possible to redisperse this mixture to a homogeneous state by stirring for 5 minutes at 40° C. Therefore the settling data for these samples are shown as 100% settled in TABLE I below. The gelatin content of these samples was 0.16 weight %.

### Invention Example 1

### Settling of Product With Br-TAI Stored at 40° C. for 5 Days

A 100 ml portion of the silver chloride dispersion prepared as described above was redispersed by stirring at 40° C. for 5 minutes, a 2.1 ml solution containing 0.055 g of (1,2,4)triazolo(1,5-a)pyrimidin-7-ol, 6-bromo-5-methyl-(Br-TAI) was added, and the mixture was stirred thereafter for 5 minutes at 40° C. The mixture was subsequently stored unstirred in an oven at 40° C. for 5 days before redispersal and testing for settling as described in Comparative Example 1. The settling data for these samples are shown in TABLE I below. The gelatin content of these samples was 0.61 weight %.

### Invention Example 2

### Settling of Product With Urazole Stored at 40° C. for 5 Days

A 100.0 ml portion of the silver chloride dispersion prepared as described above was dispersed by stirring at 40° C. for 5 minutes, a 6.2 ml solution containing 0.055 g of urazole was added, and the mixture was stirred thereafter for 5 minutes at 40° C. The mixture was subsequently stored unstirred in an oven at 40° C. for 5 days before redispersal and testing for settling as described in Comparative Example 1. The settling data for these samples are shown in TABLE I below. The gelatin content of these samples was 0.58 weight %.

### Invention Example 3

### Settling of Product With Uric Acid Stored at 40° C. for 5 Days

A 75.0 ml portion of the silver chloride dispersion prepared as described above was dispersed by stirring at 40° C. for 5 minutes, a 42.2 ml solution containing 0.055 g of uric acid, dissolved using several drops of dilute sodium hydroxide, was added, and the mixture was stirred thereafter for 5 minutes at 40° C. The mixture was subsequently stored unstirred in an oven at 40° C. for 5 days before redispersal and testing for settling as described in Comparative Example 1. The settling data for these samples are shown in TABLE I below. The gelatin content of these samples was 0.40 weight %.

### Invention Example 4

### Settling of Product With CN-BZT Stored at 40° C. for 5 Days

A 65.8 ml portion of the silver chloride dispersion prepared as described above was dispersed by stirring at 40° C. for 5 minutes, a 183.5 ml solution containing 0.036 g of 6-chloro-4-nitro-1H-benzotriazole (CN-BZT), dissolved using several drops of dilute sodium hydroxide, was added, and the mixture was stirred thereafter for 5 minutes at 40° C. The mixture was subsequently stored unstirred in an oven at 40° C. for 5 days before redispersal and testing for settling as described in Comparative Example 1. The settling data for these samples are shown in TABLE I below. The gelatin content of these samples was 0.16 weight %.

### Invention Example 5

### Settling of Product With TAI Stored at 40° C. for 5 Days

A 65.8 ml portion of the silver chloride dispersion prepared as described above was redispersed by stirring at 40° C. for 5 minutes, a 183.5 ml solution containing 0.036 g of (1,2,4)triazolo(1,5-a)pyrimidin-7-ol, 5-methyl-, sodium salt (TAI) was added, and the mixture was stirred thereafter for 5 minutes at 40° C. The mixture was subsequently stored unstirred in an oven at 40° C. for 5 days before redispersal and testing for settling as described in Comparative Example 1. The settling data for these samples are shown in TABLE I below. The gelatin content of these samples was 0.16 weight %.

### Invention Example 6

### Settling of Product With SMe-TAI Stored at 40° C. for 5 Days

A 100 ml portion of the silver chloride dispersion prepared as described above was dispersed by stirring at 40° C. for 5 minutes, a 1.9 ml solution containing 0.055 g of (1,2,4)triazolo(1,5-a)pyrimidin-7-ol, 5-methyl-2-(methylthio)-(SMe-TAI) was added, and the mixture was stirred thereafter for 5 minutes at 40° C. The mixture was subsequently stored unstirred in an oven at 40° C. for 5 days before redispersal and testing for settling as described in Comparative Example 1. The settling data for these samples are shown in TABLE I below. The gelatin content of these samples was 0.61 weight %.

### Invention Example 7

### Settling of Product With Me-BZT Stored at 40° C. for 5 Days

A 100 ml portion of the silver chloride dispersion prepared as described above was dispersed by stirring at 40° C. for 5 minutes, a 6.0 ml solution containing 0.055 g of methyl-1H-benzotriazole (Me-BZT), dissolved using several drops of dilute sodium hydroxide, was added, and the mixture was stirred thereafter for 5 minutes at 40° C. The mixture was subsequently stored unstirred in an oven at 40° C. for 5 days before redispersal and testing for settling as described in Comparative Example 1. The settling data for these samples are shown in TABLE I below. The gelatin content of these samples was 0.58 weight %.

### Comparative Example 9

### Settling of Product With Uracil Stored at 40° C. for 5 Days

A 100.0 ml portion of the silver chloride dispersion prepared as described above was dispersed by stirring at 40° C. for 5 minutes, a 10.0 ml solution containing 0.055 g of uracil was added, and the mixture was stirred thereafter for 5 minutes at 40° C. The mixture was subsequently stored unstirred in an oven at 40° C. for 5 days before redispersal and testing for settling as described in Comparative Example 1. The settling data for these samples are shown in TABLE I below. The gelatin content of these samples was 0.56 weight %.

### Comparative Example 10

### Settling of Product With Succinimide Stored at 40° C. for 5 Days

A 100.0 ml portion of the silver chloride dispersion prepared as described above was dispersed by stirring at 40° C. for 5 minutes, a 6.2 ml solution containing 0.055 g of succinimide was added, and the mixture was stirred thereafter for 5 minutes at 40° C. The mixture was subsequently stored unstirred in an oven at 40° C. for 5 days before redispersal and testing for settling as described in Comparative Example 1. The settling data for these samples are shown in TABLE I below. The gelatin content of these samples was 0.58 weight %.

The following TABLE I contains the settling data for Invention Examples 1-7 and Comparative Examples 1-10. The settling is evaluated by comparing the silver assay results for samples taken from the top of the aliquot at time=0 or later, to the theoretical silver concentration, such that percent settle is defined by the expression (Theoretical [Ag]−Silver Assay(time))/(Theoretical [Ag]))×100.

TABLE I

| | | | | Additive | Additive | | | | | |
|---|---|---|---|---|---|---|---|---|---|---|
| | | | 40° C./ | Amount | Amount | Theoretical | | | | |
| | | Additive | 5 days | (mg/g | (mmol/mol | [Ag]* Ag | % Settle | | | |
| Example | Additive | pKa | Storage | Ag) | Ag) | mol/kg | t = 0 min | t = 10 min | t = 30 min | t = 60 min |
| Comparative 1 | None | | No | 0 | 0.0 | 0.29 | 0 | 1.1 | 0 | 0 |
| Comparative 2 | None | | Yes | 0 | 0.0 | 0.29 | 0 | 49 | 49 | 49 |
| Comparative 3 | None, pH adjust | | No | 0 | 0.0 | 0.28 | 0 | 0 | 0 | 0 |
| | to 5.6 | | | | | | | | | |
| Comparative4 | None, pH adjust | | Yes | 0 | 0.0 | 0.29 | 0 | 23 | 23 | 23 |
| | to 5.6 | | | | | | | | | |
| Comparative 5 | None (water | | No | 0 | 0.0 | 0.075 | 0 | 0 | 0 | 0 |
| | dilution) | | | | | | | | | |
| Comparative 6 | None (water | | Yes | 0 | 0.0 | 0.077 | 18 | 45 | 48 | 48 |
| | dilution) | | | | | | | | | |
| Comparative 7 | Saccharin | 1.8 | Yes | 18.2 | 10.7 | 0.26 | 0 | 52 | 52 | 52 |
| Comparative 8 | APMT | 3.3 | Yes | 18.2 | 8.4 | 0.077 | ~100** | ~100** | ~100** | ~100** |
| Invention 1 | Br-TAI | 4.8 | Yes | 18.2 | 8.6 | 0.27 | 0 | 0 | 1 | 1 |
| Invention 2 | Urazole | 5.8 | Yes | 18.2 | 19.4 | 0.27 | 0 | 4.1 | 4.5 | 4.8 |
| Invention 3 | Uric acid | 5.8 | Yes | 18.2 | 11.7 | 0.18 | 0 | 0 | 0 | 0 |
| Invention 4 | CN-BZT | 6.0 | Yes | 18.2 | 9.9 | 0.077 | 0 | 0 | 0.4 | 0.4 |
| Invention 5 | TAI | 6.2 | Yes | 18.2 | 11.4 | 0.077 | 0 | 0 | 0 | 2.2 |
| Invention 6 | SMe-TAI | 6.2 | Yes | 18.2 | 10.0 | 0.28 | 0 | 0 | 0 | 1 |
| Invention 7 | Me-BZT | 8.0 | Yes | 18.2 | 14.8 | 0.27 | 0 | 0 | 0 | 0 |
| Comparative 9 | Uracil | 9.2 | Yes | 18.2 | 17.5 | 0.26 | 0 | 50 | 50 | 50 |
| Comparative 10 | Succinimide | 9.4 | Yes | 18.2 | 19.8 | 0.27 | 0 | 45 | 45 | 46 |

*Accounts for small amount of water loss during 40° C./5 day storage
**Did not redisperse so settling was not measured

Examination of the % Settle results shown above for Comparative Example 1 indicates that there are no significant settling problems for the silver chloride dispersion when it was redispersed and tested within a few hours of the preparation of the dispersion. Examination of the % Settle results for Comparative Example 2, however, indicates that severe settling occurred when the sample was redispersed and tested after storage at 40° C. for 5 days. In this case, the silver concentrations of the samples taken from the top of the redispersed aliquot were decreased by 49% after time intervals of 10-60 minutes. Comparison of the % Settle results for Comparative Examples 3 and 4 indicates a relative reduction in the settling (23%) for the pH adjusted dispersion when redispersed and tested after storage at 40° C. for 5 days. However, we note once again that an objectionable amount of discoloration was observed after storage at 40° C. for 5 days for this pH=5.6 dispersion. Examination of the % Settle results shown above for Comparative Example 5 indicates that there were no settling problems for the silver chloride dispersion diluted to 0.16 weight % gelatin. However, results for Comparative Example 6 indicate severe settling (45-48% for 10-60 minute intervals) for the silver chloride dispersion diluted to 0.16 weight % gelatin when redispersed and tested after storage at 40° C. for 5 days. In addition, the time=0 sample for Comparative Example 6 is evidence that significant settling (18%) occurred during the brief period between when the stirring was stopped and the 10 ml portion was removed from the top of the aliquot, suggesting that the initial settling occurs more quickly when the gelatin content is reduced to 0.16 weight % in comparison to results for Comparative Example 2 where the gelatin content was 0.62 weight %. Thus, a comparison of the results in Comparative Examples 5 and 6 show that without the additive, mere dilution may help with redispersability before storage but after storage, mere dilution is not helpful.

Comparison of the % Settle results for Comparative Examples 7 and 2 indicates that the addition of saccharin (pKa=1.8) was ineffective in reducing the severe settling when redispersal and testing follows storage at 40° C. for 5 days. Comparison of the % Settle results for Comparative Examples 8 and 6 indicates that the addition of APMT (pKa=3.3) was ineffective in reducing the severe settling when redispersal and testing followed storage at 40° C. for 5 days.

In marked contrast, comparison of the % Settle results for Invention Examples 1-7, wherein the Br-TAI (pKa=4.8), urazole (pKa=5.8), uric acid (pKa=5.8), CN-BZT (pKa=6.0), TAI (pKa=6.2), SMe-TAI (pKa=6.2), Me-BZT (pKa=8.0) additives were present; to the data from Comparative Examples 2 and 6 indicates that addition of N-heterocyclic acids with a pKa in the range of about 4-8.6 effectively reduced or eliminated the severe settling of silver chloride dispersions when redispersal and testing followed storage at 40° C. for 5 days.

In contrast again, comparison of the % Settle results for Comparative Examples 9 and 10, wherein uracil (pKa=9.2) and succinimide (pKa=9.4) were present, to the data from Comparative Examples 2 and 6 indicates that addition of nitrogen acids with a pKa above about 9.0 are ineffective in reducing the severe settling of silver chloride dispersions when redispersal and testing followed storage at 40° C. for 5 days.

In summary, the % Settle results shown in TABLE I above indicate that severe settling occurs when aqueous silver chloride dispersions containing between about 0.16-0.62 weight % gelatin are redispersed and tested following storage at 40° C. for 5 days. Addition of the N-heterocyclic acids with a pKa in the range of about 4-8.6, such as Br-TAI, urazole, uric acid, CN-BZT, TAI, SMe-TAI or Me-BZT, was remarkably effective in reducing or eliminating the severe settling of aqueous silver chloride dispersions containing gelatin when redispersal and testing followed storage at 40° C. for 5 days.

It is also apparent that mere dilution of the composition (for example, in Comparative Example 5) is insufficient to provide shelf life stability.

Comparative Examples 11-19 are additional experiments that examined the impact of various organic acids, surfactants, sugars, and inorganic salts upon the settling characteristics of aqueous silver chloride dispersions containing 0.16 weight % gelatin that were redispersed and tested for settling after storage at 40° C. for 5 days. Each of these nine compositions were prepared as described above for Comparative Example 8 except that the APMT additive was replaced with the compounds listed in TABLE II below.

TABLE II

| | | 40° C./5 day | % Settle |
|---|---|---|---|
| Example | Additive | Storage | t = 60 min |
| Comparative 11 | Citric Acid | Yes | 100%* |
| Comparative 12 | 0.1 molar Sucrose | Yes | >30% |
| Comparative 13 | 2% TRITON ® X-100 | Yes | >30% |
| Comparative 14 | 1% BURCOWET ® | Yes | >30% |
| | surfactant | | |
| Comparative 15 | 0.1% Sodium | Yes | >30% |
| | Dodecylsulfate | | |
| Comparative 16 | Magnesium Chloride | Yes | >30% |
| Comparative 17 | Tetraethyl Ammonium | Yes | >30% |
| | Chloride | | |
| Comparative 18 | Ammonium Nitrate | Yes | >30% |
| Comparative 19 | Ammonium Sulfate | Yes | >30% |

*Sample did not redisperse

The settling results shown in TABLE II indicate that none of the additives used in Comparative Examples 11-19 provided sufficient reduction in settling to be of practical use. In particular, the result for Comparative Example 17 indicates that an alkyl ammonium halogenide additive (for example, tetraethyl ammonium chloride) was not sufficiently effective to reduce settling in the composition to be of practical use.

The invention has been described in detail with particular reference to certain preferred embodiments thereof, but it will be understood that variations and modifications can be effected within the spirit and scope of the invention.

1. A composition comprising at least 50 weight % water, silver halide particles, gelatin, and an additive that includes one or more N-heterocyclic acids at least one of which has a pKa of from about 4 to about 9, wherein said composition is substantially free of organic solvents.

2. The composition of claim 1 wherein said N-heterocyclic acid is an azole.

3. The composition of claim 1 wherein said N-heterocyclic acid is a triazole.

4. The composition of claim 1 wherein said N-heterocyclic acid is a diazole.

5. The composition of claim 1 wherein said N-heterocyclic acid has a pKa of from about 4.5 to about 8.5.

6. The composition of claim 1 wherein said N-heterocyclic acid is one or more of the following compounds:
tetraazaindene, bromo-tetraazaindene, S-methyl-tetraazaindene, urazole, uric acid, benzotriazole, methylbenzotriazole, and chloronitrobenzotriazole.

7. The composition of claim 1 wherein said additive is present in an amount of from about 0.1 to about 5 weight % relative to the amount of silver.

8. The composition of claim 1 wherein gelatin is present in an amount of less than 1.3 weight %.

9. The composition of claim 1 wherein said silver halide particles are predominantly silver chloride.

10. The composition of claim 1 wherein said silver halide particles are present in an amount of from about 1×10⁻⁷ to about 20% by weight.

11. The composition of claim 1 wherein said silver halide particles are present in an amount of from about 1×10⁻⁴ to about 10% by weight.

12. The composition of claim 1 wherein said silver halide particles are predominantly less than 1 μm in diameter.

13. The composition of claim 1 that does not substantially gel or solidify at 25° C.

14. The composition of claim 1 comprising at least 90 weight % water, silver chloride particles in an amount of from about 0.001 to about 4 weight %, gelatin in an amount of from about 0.15 to about 1 weight %, and an additive that includes one or both of uric acid and methylbenzotriazole in an amount of from about 0.5 to about 2 weight % relative to the amount of silver, wherein said composition is substantially free of organic solvents.

15. A composition consisting essentially of water, silver halide particles, gelatin, and an additive that includes one or more N-heterocyclic acids at least one of which has a pKa of from about 4 to about 9.

16. The composition of claim 15 wherein said N-heterocyclic acid is an azole.

17. The composition of claim 15 wherein said N-heterocyclic acid is one or more of the following compounds:
tetraazaindene, bromo-tetraazaindene, S-methyl-tetraazaindene, urazole, uric acid, benzotriazole, methylbenzotriazole, and chloronitrobenzotriazole.

18. The composition of claim 15 wherein gelatin is present in an amount of less than 1 weight %.

19. The composition of claim 15 wherein said silver halide particles are predominantly silver chloride and are present in an amount of from about 1×10⁻⁷ to about 10% by weight.

20. A method of providing an antimicrobial coating comprising:
coating a fiber, fabric, or substrate with the composition of claim 1, and
drying the coated fiber, fabric, or substrate.

21. A fiber, fabric, or substrate having a dried antimicrobial coating provided from the composition of claim 1.
